(19) United States
(12) Patent Application Publication
(10) Pub. No.: US 2026/0218274 A1
(43) Pub. Date: Jul. 30, 2026

(54) DEVICE FOR DETECTING A TARGET NUCLEIC ACID SEQUENCE

(71) Applicant: UNIVERSITEIT TWENTE, Enschede (NL)

(72) Inventors: Jeanne Elisabeth VAN DONGEN; Jan Cornelis Titus EIJKEL; Jurriaan HÜSKENS; Loes Irene SEGERINK

(21) Appl. No.: 19/112,537

(22) PCT Filed: Sep. 19, 2023
(86) PCT No.: PCT/EP2023/075706

§ 371 Date: Mar. 17, 2025

(30) Foreign Application Priority Data
Sep. 19, 2022 (NL) 2033069

(51) Int. Cl. C12Q 1/6825; C12N 9/22; C12N 15/11; C12Q 1/6818; C12Q 1/6886

(52) U.S. Cl. CPC C12Q 1/6825; C12N 9/226; C12N 15/11; C12Q 1/6818; C12Q 1/6886; C12N 2310/20; C12N 2320/10; C12Q 2600/154

(57) ABSTRACT

The present invention relates to device for detecting at least one target nucleic acid sequence. The device comprises a surface which carries at least one activatable protein which is activated in the presence of the at least one target nucleic acid sequence; one or more reporter constructs which provide a detectable signal upon activation of the activatable protein, wherein the at least one activatable protein and/or the one or more reporter constructs are immobilized, optionally via a linker, to one or more moieties capable of moving along the surface.

### TECHNICAL FIELD

The present invention relates to a nucleic acid detection technique preferably based on CRISPR/Cas sensing. The technique is suitable for detecting biomarkers in diseases such as cancer.

### BACKGROUND OF THE INVENTION

Reliable, accurate, and sensitive nucleic acid detection is needed for preventing late-stage disease development, such as tumour metastasis or severe pathogen infections.

Traditionally, amplification-based methods like polymerase chain reaction (PCR) have been considered the “gold standard” regarding nucleic acid detection.

CRISPR/Cas sensing is gaining ground as a simple and easy-to-use technique to detect DNA or RNA of interest. CRISPR sensing techniques generally use a ribonucleotide protein (RNP) that consists of a CRISPR/Cas effector protein supplemented with an RNA sequence (crRNA). This RNP complex can typically target the sequence of interest at concentrations in the order of pM within one hour or so (Chen et al. Science. 2018 Apr. 27; 360 (6387): 436-439).

A limitation is that cell-free nucleotide concentrations in liquid biopsies are typically present in the fM range (Marrugo-Ramírez et al. 2018 Sep. 21; 19 (10): 2877). Therefore, preamplification methods are generally combined with CRISPR sensing to lower the detection limit further. However, while preamplification decreases the detection limit, it also causes longer assay times and regularly induces amplification errors that affect the CRISPR assay's selectivity. Furthermore, these amplification methods make the quantitative results obtained by CRISPR sensing qualitative. This removes information about the starting concentration, whereas medical applications require absolute target quantification to precisely determine expression levels in, for example, liquid biopsies.

Microfluidic confinement methods have been proposed to lower the detection limit of CRISPR assays to low fM concentration, however they are associated with undesirable binding of RNP complexes to target sequences (e.g. when mixing target and RNP before partitioning) or suboptimal quantification (e.g. in case of poor mixing).

It is an objective of the present invention to overcome at least one of the above-mentioned or other problems.

### SUMMARY OF THE INVENTION

In an aspect, the present disclosure relates to a method and device which make use of a (2D) sensor surface functionalized with an activatable protein, preferably an activatable protein with tuneable trans-cleavage activity, more preferably an RNP complex comprising CRISPR/Cas 12 effector protein supplemented with a CRISPR RNA sequence (crRNA). The method and device preferably also makes use of reporter constructs which provide a detectable signal upon activation of the protein. By exploiting the lateral diffusion of the activatable protein (and the reporter constructs) along the sensor surface, a single activated protein can induce a detectable signal in multiple reporter constructs, in this way enabling single molecule sensing which allows drastically lowering the detection limit of nucleic acid detection assays. The current method and device further overcome the limitations of microfluidic confinement known in the art, which are associated with undesirable binding of activatable protein (e.g. RNP complexes) or suboptimal quantification.

In an aspect, the present disclosure relates to a device for detecting at least one target nucleic acid sequence, wherein the device comprises a surface which carries
at least one activatable protein which is detectably activated in the presence of the at least one target nucleic acid sequence;
preferably one or more reporter constructs which provide a detectable signal upon activation of the activatable protein,
   wherein the at least one activatable protein and/or the one or more reporter constructs are immobilized, optionally via a linker, to one or more moieties capable of moving along the surface.

The activatable protein is preferably a CRISPR/Cas effector protein supplemented with a CRISPR RNA sequence (crRNA).

In an aspect, the present disclosure relates to a use of the device as disclosed herein for detecting a nucleic acid target sequence, preferably in the diagnosis of cancer.

In a further aspect, the present disclosure relates to a method for detecting at least one target nucleic acid sequence, the method comprising
i. applying a liquid sample comprising the at least one target nucleic acid sequence to the surface of the above-mentioned device; and
ii. detecting activation of the activatable protein, preferably by measuring the detectable signal provided by the one or more reporter constructs.

### DETAILED DESCRIPTION OF THE INVENTION

The present disclosure pertains to a device for detecting at least one target nucleic acid sequence, wherein the device comprises a surface which has or carries
at least one activatable protein which is detectably activated in the presence of the at least one target nucleic acid sequence;
preferably one or more reporter constructs, such as at least 2, 5, 10, 25 reporter constructs, which provide a detectable signal upon activation of the activatable protein.

In an aspect, the at least one activatable protein is immobilized to or on the surface, optionally via a linker and preferably to one or more moieties capable of moving (laterally) along the surface. In addition or alternatively, the one or more reporter constructs are immobilized to or on the surface, optionally via a linker and preferably to one or more moieties capable of moving (laterally) along (i.e. over or across) the surface.

The at least one target nucleic acid sequence may be at least one double-stranded nucleic acid sequence and/or at least one single-stranded nucleic acid sequence. The device may be in combination with the at least one target nucleic acid sequence. The at least one target nucleic acid sequence as according to the present disclosure may have a length of between 10 bp and 10 kb, preferably between 20 bp and 1 kb. In an embodiment, the target nucleic acid sequence is single-stranded ribonucleic acid (RNA). In an embodiment, the target nucleic acid is double-stranded RNA. The RNA as disclosed herein encompasses messenger RNA (mRNA), transfer RNA (IRNA) and/or ribosomal RNA (rRNA). In an embodiment, the target nucleic acid is single-stranded deoxyribonucleic acid (DNA). In an embodiment, the target nucleic acid is double-stranded DNA. The DNA as disclosed herein encompasses circulating tumour DNA (ctDNA) and cell free DNA (cfDNA).

The target nucleic acid sequence as disclosed herein preferably comprises a biomarker (in disease), more preferably a cancer biomarker. The term “biomarker” in the context of the current disclosure means a marker which presence or absence (e.g. in a body tissue or a body fluid) indicates a normal or abnormal process, or indicates a condition or disease (e.g. cancer). In addition or alternatively, a biomarker (level) may indicate efficacy of treatment of a disease or condition (e.g. cancer). The term “biomarker” in the context of the current disclosure encompasses molecular markers and signature molecules. In an embodiment, the biomarker represents epigenetic changes and/or post-translational modifications associated with the development of disease, preferably cancer. One of these epigenetic changes includes DNA (hyper)methylation. Hence, the target nucleic acid sequence of the present disclosure is preferably associated with development, progression and/or treatment of disease, preferably cancer. The cancer in the context of the current invention preferably is one or more of breast cancer, lung cancer, bronchus cancer, prostate cancer, colon cancer, rectum cancer, melanoma, bladder cancer, non-Hodgkin lymphoma, kidney cancer, renal pelvis cancer, endometrial cancer, leukaemia, pancreatic cancer, thyroid cancer, and liver cancer.

The target nucleic acid sequence preferably is comprised in a tissue sample and/or body fluid sample (e.g. blood, blood plasma or urine) obtained from a subject, such as selected from the group consisting of a tissue biopsy, urine, blood, saliva, serum, blood plasma, interstitial fluid, synovial fluid, transudate, pus, breast milk, and semen, more preferably urine. The device according to the present disclosure may be in combination with said tissue sample and/or body fluid sample.

The target nucleic acid sequence preferably comprises one or more sites which can undergo (or has undergone) post-translational modification, preferably one or more of (hyper)methylation, glycosylation, acetylation, neddylation, phosphorylation, prenylation, SUMOylation, and ubiquitination, and wherein said post-translational modification is associated with disease and/or is a biomarker according to the present disclosure. Preferably, the target nucleic acid can undergo (or has undergone) CpG methylation (i.e. methylated cytosines in CpG dinucleotide).

In an embodiment, the activatable protein undergoes a “conformational change”, meaning a change of the protein's tertiary structure in response to external factors (e.g. pH, temperature, solute concentration) or after association with a (target) ligand or nucleic acid. The conformational change preferably leads to enzymatic activity, such as cleavage activity as disclosed herein. In an embodiment, the activatable protein provides, upon activation, (increased) cleavage activity of nucleic acid, preferably single-stranded nucleic acid (ssRNA, ssDNA), wherein the cleavage may be cis- (i.e. cleavage of the target nucleic acid sequence) and/or trans-cleavage (i.e. cleavage of non-target nucleic acid sequence). The conformational change preferably leads to a detectable signal, such as provided by the one or more reporter constructs as disclosed herein.

In a preferred embodiment, the activatable protein is a Cas (effector) protein. The (CRISPR/) Cas protein may be of the class I, class II and/or class III, and/or may be chosen from the group consisting Cas 1-14. In an embodiment, the Cas protein is Cas 12, e.g. Cas12a (such as FnCas12a, Cas12a, or LbCas12a) or Cas 12b. In an embodiment, the Cas protein is Cas13, e.g. Cas 13a (i.e. C2c2), Cas13b, Cas13c or Cas13d. In an embodiment, the Cas protein is Cas14, e.g. Cas 14a, Cas 14b, or Cas 14c. In an embodiment, the Cas12a is FnCas12a, LbCas12a. In an embodiment, the (CRISP/) Cas protein is an Alt-R (CRISP/) Cas protein, such as ALT-R Cas12a.

In a preferred embodiment, the at least one activatable protein is supplemented with an RNA sequence complementary to the at least one target nucleic acid sequence, e.g. CRISPR (crRNA), meaning that the RNA sequence and the target sequence can undergo base pairing.

In a preferred embodiment, the activatable protein is a ribonucleotide protein (RNP), e.g. a (CRISPR/) Cas protein supplemented with an RNA sequence (e.g. or RNA) which is preferably complementary to the at least one target nucleic acid sequence as disclosed herein, meaning that the RNA sequence and the target sequence can undergo base pairing. The Cas protein and/or the RNP preferably (further) comprises or is supplemented with a guide RNA (gRNA) which recognizes the at least one target nucleic acid sequence and directs the Cas protein to make double-strand breaks in nucleic acids.

The term “activation” (of a protein) preferably means that said protein undergoes a conformational change and/or obtains increased (enzymatic) activity, such as cleavage activity. The term “activatable protein” thus means that said protein is capable of undergoing conformational change and/or obtaining increased (enzymatic) activity, such as cleavage activity, particularly upon recognition/binding of the at least one target nucleic acid sequence. In an embodiment, a Cas protein or RNP is activated following base-pairing of the at least one target nucleic acid sequence and the complementary RNA sequence it is supplemented with.

In an embodiment, the RNP undergoes a structural change upon hybridization between the crRNA and a complementary target nucleic acid sequence; this structural change may reveal the catalytic site of the protein and unleashes the enzymatic activity such as trans-cleavage activity of the protein (e.g. Cas protein such as Cas12, Cas 12a). In a preferred embodiment, activation of the activatable protein provides said protein of trans-cleavage activity.

The term “capable of moving along the surface” when describing movement of a moiety as described herein can mean rotational, lateral movement and/or diffusion (such as in a lipid bilayer), or transverse movement (such as between lipid bilayers), The “moving” as used herein encompasses diffusion. Lipids in a lipid bilayer are generally free to move laterally (unless restricted by certain interactions). Preferably, the term “capable of moving along the surface” when describing movement of a moiety in the context of the current invention means lateral diffusion. The lateral movement and/or diffusion encompasses “hop diffusion”, meaning that a lipid moiety stays in one region (in a lipid bilayer) for a short period before hopping to another location.

In a preferred embodiment, the one or more moieties capable of moving along the surface (spontaneously) form a bilayer in an aqueous environment. In a preferred embodiment, the one or more moieties capable of moving along the surface is one or more lipid molecule. In an embodiment, the one or more moieties capable of moving along the surface is one or more amphipathic molecule such as cholesterol or such as a hydrophobic molecule. In an embodiment, the one or more moieties capable of moving along the surface has/have a hydrophilic moiety (head) and a hydrophobic moiety (tail). The one or more moieties according to the present disclosure may be functionalized and/or may have a functional group allowing coupling of the at least one activatable protein and/or the one or more reporter constructs. In an embodiment, the one or more moieties capable of moving along the surface is a phospholipid. The phospholipid as disclosed herein may be chosen from the group consisting of a phosphatidic acid (phosphatidate) (PA), a phosphatidylethanolamine (cephalin) (PE), a phosphatidylcholine (lecithin) (PC), a phosphatidylserine (PS), a phosphoinositide, a phosphatidylinositol (PI), a phosphatidylinositol phosphate (PIP), a phosphatidylinositol a bisphosphate (PIP2), and a phosphatidylinositol trisphosphate (PIP3).

In a preferred embodiment, the device comprises a surface carrying a layer of said one or more moieties, preferably a (first, top) layer of lipids and/or a (second, bottom) layer of lipids, wherein preferably the one or more moieties capable of moving along the surface as disclosed herein are comprised in the first and/or second layer of lipids.

In a preferred embodiment, at least part of the lipids in the first and the second layer of lipids have a hydrophilic moiety and a hydrophobic moiety thereby at least partially forming a lipid bilayer. For example, the surface carries a lipid monolayer or lipid bilayer, preferably a lipid bilayer comprising a first layer of lipids and a second layer of lipids and wherein the one or more moieties capable of moving along the surface are comprised in the first and/or second layer of lipids. Alternatively, the surface may be a hydrophobic surface and/or carrying a lipid monolayer. This allows pointing of the lipid's hydrophobic tails to the surface, and the lipid's hydrophilic headgroups to the solution on top, thereby having identical functionality.

In an embodiment, the surface as disclosed herein is comprised in or on a solid (support), which preferably is comprised in the device as disclosed herein. In an embodiment, the surface as disclosed herein is (at least partially) hydrophilic (after carrying hydrophobic moieties such as a lipid bilayer). In an embodiment, the surface as disclosed herein is glass and/or silica. The hydrophilicity of the surface may be achieved by coating with hydrophilic chemical moieties (e.g. hydrophilic polymer such as PEG) or nanoparticles (e.g. by TiO₂, SiO₂, Mg(OH)₂, ZnO, carbon nanotubes, boehmite nanoparticles). Hydrophilic means the moiety or surface attracts water and/or does not repel water and/or comprises polar (surface) molecule(s) (or comprises (surface) molecules having a solubility in water of above 1000, 100, 10, 5, 1 mg/l). Hydrophobic means repelling water and/or not attracting water and/or comprising non-polar (surface) molecules (or comprises (surface) molecules having a solubility in water of below 1000, 100, 10, 5, 1 mg/l).

The term “immobilization to a moiety” (of a molecule such as a protein or nucleic acid sequence) as used herein means that a molecule is attached to the moiety in a reversible or irreversible manner. The molecule may directly bind to said moiety (e.g. a lipid) without requiring a linker. In addition or alternatively, a linker between the molecule and the moiety (e.g. a lipid molecule) may establish the immobilization. The “linker” in the context of the current disclosure preferably means a chemical linker, providing one or more bonds as disclosed herein. For example, the one or moieties may comprise a functional group allowing coupling of the at least one activatable protein and/or the one or more reporter constructs. The term “immobilization” encompasses the irreversible or the reversible binding. The immobilization in the context of the current disclosure encompasses non-specific immobilization (i.e. leading to immobilized molecules oriented in a random fashion), such as non-covalent adsorption or covalent non-specific attachment. The immobilization in the context of the current disclosure also encompasses specific immobilization (i.e. leading to uniform orientation of immobilized molecules). Preferably, the immobilization is by one or more of a chemical bond, a covalent chemical bond, a non-covalent chemical bond (such as Van der Waals forces, hydrogen bonding, electrostatic interaction, hydrophobic interaction, hydrophilic interaction, receptor-ligand interaction).

The “immobilization” may involve specific immobilization, more preferably using thiazolidine ring formation, Diels-Alder reaction, Staudinger ligation, a-oxo semicarbazone ligation, His-Ni-NTA, biotin binding to biotin-binding moiety (e.g. biotin binding to streptavidin, avidin, NeutrAvidin), native chemical ligation.

In a preferred embodiment, the immobilization involves a click chemistry reaction, such as one or more of cycloaddition reactions, such as the 1,3-dipolar family, and hetero Diels-Alder reactions; nucleophilic ring-opening reactions (e.g., epoxides, aziridines, cyclic sulfates, and so forth); carbonyl chemistry, such as the formation of oxime ethers, hydrazones, and aromatic heterocycles; in addition to carbon-carbon multiple bonds, such as epoxidation and dihydroxylation and azide-phosphine coupling (Staudinger ligation).

In a preferred embodiment, the immobilization is by one or more of a chemical bond, a covalent chemical bond, a non-covalent chemical bond and/or wherein a linker provides one or more of said bonds.

In a preferred embodiment, the immobilization is provided by a bond between biotin and a biotin-binding moiety and/or the linker is biotin or a biotin-binding moiety. The “biotin-binding moiety” is in the context of the current invention is preferably avidin/streptavidin or NeutrAvidin.

The linker can be one or more functional chemical moieties. For example, the (moveable) lipid (molecule) can be functionalized with a chemical moiety to react with a chemical moiety of the activatable protein or reporter construct resulting in attachment. Examples of functional moieties groups include, but are not limited to, amino, hydroxyl, carboxyl, carboxylate, aldehyde, ester, ether (e.g. thio-ether), amide, amine, nitrile, vinyl, sulfide, sulfonyl, siloxanes, phosphoryl, oxo, thiol, or similar chemically reactive functional groups. Additional moieties that can be used as functional groups as part of the linker, but are not limited to, maleimide, N-hydroxysuccinimide, sulfo-N-hydroxysuccinimide, nitrilotriacetic acid, activated hydroxyl, haloacetyl (e.g., bromoacetyl, iodoacetyl), activated carboxyl, hydrazide, epoxy, aziridine, sulfonylchloride, trifluoromethyldiaziridine, pyridyldisulfide, N-acyl-imidazole, imidazolecarbamate, vinylsulfone, succinimidylcarbonate, arylazide, anhydride, diazoacetate, benzophenone, isothiocyanate, isocyanate, imidoester, fluorobenzene, biotin and avidin. In a preferred embodiment, the RNA sequence complementary to the at least one target nucleic acid sequence in the context of the current disclosure is immobilized one the one or more of the moieties capable of moving along the surface. Accordingly, the RNA sequence complementary to the at least one target nucleic acid sequence can be the linker.

Preferably, the device as disclosed herein comprises one or more reporter constructs that are optionally immobilized, such as via a bond as disclosed herein or a linker providing such bond, to one or more of the (moveable) moieties (e.g. lipid(s)). The reporter constructs preferably provide a detectable signal upon activation of the activatable protein, typically downstream/caused by the conformation change in the activatable protein. For example, a change in the protein's tertiary structure in response to an external factors and/or the presence and base pairing with at least one target nucleic acid sequence may lead to (increased) enzymatic activity of the activated protein, said activity inducing the reporter construct to provide (or increase) the detectable signal. In a preferred embodiment, the binding of complementary RNA of the Cas protein/RNP leads to cleavage activity by the Cas protein/RNP, thereby cleaving a nucleic acid sequence in the reporter construct. The cleavage of the nucleic acid sequence leads to an increase in the detectable signal provided by the reporter construct(s), such as following release of label as disclosed herein, preferably a fluorophore (or quencher thereof). The presence and/or amount of the at least one target nucleic acid sequence can be correlated to the amount of fluorescence signal. The cleavage activity is preferably trans-cleavage activity, such that any single-stranded nucleic acid sequence is cleaved. In another example, the activation of the activatable protein leads to release of a chemical moiety, which can be detected by further reacting the sample with a substrate for the chemical moiety and quantifying the chemical reaction.

Preferably, the detectable signal in the reporter construct(s) is associated with the activation of the activatable protein, meaning that the signal is absent or low when said protein is not activated, but present and/or high when said protein is activated. Preferably, the signal provided by the reporter constructs is/can be correlated to the amount of target nucleic acid and/or the activation of the activatable protein.

The term “reporter construct” as used herein means any molecule that can be used to provide a detectable (preferably quantifiable) effect such as a detectable (physical) signal. Suitable reporter constructs in the context of the current invention can be or comprise a label such as a dye (e.g. a fluorescent dye or moiety, a redox label, a fluorophore), a radioactive label, a moiety allowing one or more types of bonds as disclosed herein, a hapten, a luminescent molecule or compound, a phosphorescent molecule or compound, or a (metal) nanoparticle. In addition or alternatively, a suitable reporter construct or label could be one that can provide one or more of the following signals: fluorescence, electrical, electrochemical, radioactivity, colorimetry, gravimetric analysis, X-ray diffraction or absorption, magnetic properties, enzymatic activity, quality characteristics, mass and charge.

In a preferred embodiment, activation of the activatable protein provides said protein of trans-cleavage activity and preferably the trans-cleavage activity cleaves at least one single or double-stranded nucleic acid in the reporter construct thereby providing a detectable signal. In a preferred embodiment, the reporter construct comprises a fluorophore and a quencher of the fluorophore separated by at least one single-stranded nucleic acid cleavable by trans-cleavage activity.

It is advantageous in the context of the current invention to provide a specific number of activatable proteins and reporter constructs (per partition in the case of two or more separated partitions).

Preferably, there are at least 1, 2, 3, 4, 5, 6, 7, 8, 9, 10, 15, 20, 50, 100, 1000, 10000, or 100000 activatable proteins per surface and/or partition (in the case the surface has two or more separated partitions).

In a preferred embodiment, there are one, two, or three activatable proteins per surface or partition.

Preferably there are at least 1, 2, 3, 4, 5, 10, 100, 1×10³, 1×10⁴, 1×10⁵, 1×10⁶, 1×10⁷, 1×10⁸, 1×10⁹, 1×10¹⁰, or 1×10¹² reporter constructs per device/surface and/or partition (in the case of two or more separated partitions).

In a preferred embodiment, the number of activatable proteins and the number of reporter constructs (per surface or per partition) is in a ratio of between 1:1-1:1000000, preferably 1:10-1:100000, more preferably 1:200-1:50000.

It is preferred that the device (and/or surface) according to the invention does not comprise metal particle(s), and/or not comprise metal particle(s) linked to a ligand or moiety capable of binding the target nucleic acid sequence (i.e. metal particle being between 1-1000, preferably 1-100 or 25-75 nm).

The device or surface of the current invention may have two or more partitions, such as at least 10, 20, 100, 1000, 10000, 1000000, 1000000000 separated partitions. Accordingly, the present disclosure provides a corral-type sensor, e.g. where each corral acts as an individual sensor e.g. for different targets. Each partition may for example serve to detect a different target nucleic acid sequence (e.g. biomarker) and/or comprise different activatable proteins and/or supplemented with different (crRNA) sequences complementary to the target nucleic acid sequence. The use of two or more partitions can accommodate a marker panel, wherein the level of expression of each marker or the overall marker profile can be linked to presence of disease and/or disease status. The Example of the present disclosure illustrates a marker panel comprising GHSR/MAL as DNA methylation markers, wherein the expression of both markers is considered in the diagnosis of bladder cancer.

In a preferred embodiment, the surface of the device comprises at least two (or at least 10, 20, 100, 1000, 10000, 1000000, 1000000000) separate partitions each comprising
the at least one activatable protein which is activated in the presence of the at least one target nucleic acid sequence;
the one or more reporter constructs which provide a detectable signal upon activation of the activatable protein,
   wherein the at least one activatable protein and/or the one or more reporter constructs are immobilized in each partition, optionally via a linker, to one or more moieties capable of moving along the surface.

It is preferred that the partitions, or at least 50, 60, 70, 80, 90, 91, 92, 93, 94, 95, 96, 97, 98, 99% of the partitions, contain either one or zero molecule of the target nucleic acid sequence. This allows to more precisely quantify low concentrations of target nucleic acid sequences.

It is preferred that the surface of the present disclosure is (substantially) flat.

In a preferred embodiment, the at least two separate partitions are formed by physical and/or hydrophobic enclosures thereby preventing exchange of the at least one activatable protein and/or the one or more reporter constructs between the at least two partitions. The enclosure preferably extends in a vertical orientation from a horizontally-oriented surface. The enclosure may provide a physical and/or hydrophilic/hydrophobic barrier to prevent exchange of the at least one activatable protein and/or the one or more reporter constructs between the at least two partitions.

The present disclosure further provides for a method for detecting at least one target nucleic acid sequence (preferably as disclosed herein) the method comprising
i. applying (e.g. introducing or contacting) a liquid sample (preferably as disclosed herein) comprising the at least one target nucleic acid sequence to the surface of the device (as disclosed herein); and
ii. detecting activation of the activatable protein, preferably by measuring the detectable signal provided by the one or more reporter constructs.

The term “applying” encompasses the contacting of the liquid sample with the device and/or surface. The term “applying” encompasses the dividing the liquid sample over the two or more partitions as disclosed herein.

The “subject” as disclosed herein may be any mammal, preferably a human. The subject may be healthy or diseased. The subject can be a patient, preferably a cancer patient. In a preferred embodiment, the target nucleic acid sequence is present in the liquid sample in an amount of 1×10⁻²²-1×10⁻⁶ M, or 1×10⁻¹⁸-1×10⁻¹⁰ M, or 1×10⁻¹⁴-1×10⁻¹² M.

The device and/or method of the present disclosure is preferably for in-vitro or ex-vivo use. Preferably, the device and/or method of the present disclosure is not practiced on the human or animal body. For example, a tissue or fluid of a subject is obtained (comprising the one or more target nucleic acid sequence, biomarker), whereafter the detection is performed outside of the body, such as in the laboratory. Preferably, the tissue or fluid is not introduced (back) into the subject.

In an embodiment, the device is implanted into the human body and detection of the target nucleic acid sequence/biomarker is performed in vivo. In an embodiment, the method is for in-vivo detecting the target nucleic acid sequence/biomarker.

The present disclosure also pertains to a use of the method and/or device for detecting at least one target nucleic acid sequence, preferably in the diagnosis of disease, preferably cancer. The use can be in vitro or ex vivo, and preferably is not practiced on the human or animal body. Preferably the present method and/or use does not comprise a DNA amplification step, and/or for example does not comprise a polymerase chain reaction (PCR) amplification step.

In this document and in its claims, the verb “to comprise” and its conjugations is used in its non-limiting sense to mean that items following the word are included, but items not specifically mentioned are not excluded.

In this document and in its claims, reference to an element by the indefinite article “a” or “an” does not exclude the possibility that more than one of the element is present, unless the context clearly requires that there be one and only one of the elements. The indefinite article “a” or “an” thus usually means “at least one”.

In the context of the current invention, the terms ‘to increase’ and ‘increased level’ and the terms ‘to decrease’ and ‘decreased level’ refer to the ability to significantly increase or significantly decrease or to a significantly increased level or significantly decreased level. Generally, a level is increased or decreased when it is at least 5%, such as 10%, 15%, 20%, 25%, 30%, 35%, 40%, 45%, 50% higher or lower, respectively, than the corresponding level in a control or reference. Alternatively, a level in a sample may be increased or decreased when it is statistically significantly increased or decreased compared to a level in a control or reference. The term “to reduce” may herein be used interchangeably with “to decrease”. The term “reducing” may herein be used interchangeably with “decreasing”.

The Example illustrates the different embodiments of the invention.

### FIGURE LEGENDS

FIG. 1. Schematic representation of a single well (e.g. “corral”) containing CRISPR. The proteins and fluorophore-quencher pairs can laterally diffuse across the well.

A) In the absence of a target sequence, Cas12a is in its inactivated state and cannot cleave the ssDNA between the quencher and fluorophore. B) in the presence of a target sequence, Cas12a will cleave the ssDNA between the quencher and fluorophore, increasing the fluorescence.

FIG. 2. Design of the trans-cleavage reporter and its immobilization of the supported lipid bilayers (SLB). Biotinylated ssDNA containing a green fluorescent molecule can interact with the Streptavidins (SAvs) immobilized on the SLB. Next, the partly complementary ss-DNA containing a quencher can hybridize. This ssDNA strand has an overhang of 5 nts, allowing the Cas12a RNP to cleave between the fluorophore and quencher upon target recognition.

FIG. 3. Quartz crystal microbalance with dissipation (QCM-D) measurements of the assembly of the sensor. Three individual measurements of the sensor assembly included the supported lipid bilayer (SLB) formation (I), changing buffer from 0.9×PBS to 1×PBS (II) Streptavidin (Sav) coating (III), ssDNA-biotin immobilization (IV), and ssDNA-quencher hybridization (V). All white parts resemble washing with 1×PBS (after II).

FIG. 4. Trans-cleavage activity of Cas 12a supplemented with differently modified crRNAs. A) Trans-cleavage activity for the extended crRNA B) Schematics of (on top) the 5′ biotin samples, the conventional crRNA with an extra 7 nt poly-U tail on the 5′ end followed by a biotin modification, and (on the bottom) a 3′ biotin sample, which contains the 7 nt poly U tail between the conventional sequence and the biotin modification.

FIG. 5. Quartz crystal microbalance with dissipation (QCM-D) measurements of the assembly of the sensor. A) Steps to immobilize 3′ and 5′ biotinylated crRNAs. (I) supported lipid bilayer (SLB) formation (II) Streptavidin (SAv) coating (III) crRNA-biotin immobilization. B) Cas12a immobilization step, resulting in RNP formation on the SLB. All white parts resemble washing with 1×PBS.

FIG. 6. Trans-cleavage activity of Cas12a supplemented with 3′ and 5′ biotinylated crRNAs. As measured towards a fluorophore-quencher pair in solution. A plate reader was used to monitor the increase in fluorescence over time with 5 minute time interval.

### EXAMPLE

The current example provides a 2D confinement system where a sensor surface is functionalized with a RNP complex and a fluorophore-quencher pair. Such a system utilizes the immobilization of both RNP and fluorophore-quencher to the sensor. By exploiting the unique lateral diffusion within supported lipid bilayers boxed by hydrophilic enclosures such as (hydrophilic) metal lines, 2D diffusion of reporter and CRISPR/Cas protein can be enabled within the microwell. This diffusion allows a single CRISPR/Cas protein to cleave multiple reporters, in this way enabling single molecule sensing. The current example shows the successful functionalization of the different components of this sensor, including the fluorophore-quencher reporter pair, as well as the anchoring of the Cas12a protein via its crRNA sequence to the SLB.

### Methods

### Oligonucleotides

Eurofins Genomics synthesized the DNA oligonucleotides. All crRNA fragments were Alt-R® A.s. Cas12a crRNAs, or variants thereof, synthesized by Integrated DNA Technologies (IDT, Coralville IA).

### Cas12a Ribonucleoprotein Complex Formation

ALT-R CRISPR-Cas12a (Cpf1) Ultra (Integrated DNA technologies) and custom crRNA were mixed in a ratio of 1:2 for 30 minutes at room temperature to form ribonucleotide protein (RNP) complexes for solution experiments. The assembled complex was then diluted 100 times in RNAse-free water (MACHEREY-NAGEL) and stored in the freezer at −20° C. until further use.

### Cas12a Collateral Cleavage Assay

In a total volume of 70 μl, 1× buffer, 20 nM of RNP complex, and 1000 nM of fluorophore-Quencher “reporter” DNA (Eurofins Genomics) were mixed with different concentrations of target dsDNA. Mass screening experiments were performed in a black polypropylene 384 well plate (Corning®), where each well was filled with 20 μl of the reaction mixture, allowing triplicates of the total reaction volume. In these mass screening experiments, a MasterMix was prepared to contain the RNP, buffer, and reporter DNA. Both the MasterMix and reaction mixture were pipetted on ice, and the reaction mixture was transferred to a pre-heated, black 384-well plate to inhibit reactions until the plate was transferred to the plate reader. All experiments were performed at 38° C. unless otherwise stated.

Large Unilamellar Vesicles (LUV) and Supported Lipid Bilayer (SLB) formation 1,2-dioleoyl-sn-glycero-3-phosphoethanolamine-N-(cap biotinyl) (Biotin-DOPE) and 1,2 dioleoyl-sn-glycero-3-phosphocholine (DOPC) in chloroform (CHCl3) (Avanti Polar Lipids, Inc) were diluted further with CHCl3 to obtain stock solutions with concentrations of 0.2 mg/mL and 10 mg/mL, respectively. Texas Red DHPE (TRDHPE) (Thermo Fisher Scientific) is purchased as a solid and dissolved in methanol to obtain a stock solution with a 0.2 mg/mL concentration. Streptavidin Alexa Fluor 488 (SAv-488) (Thermo Fisher Scientific) was dissolved in PBS to obtain a stock solution with a concentration of 0.2 mM. Phosphate buffer saline (PBS) tablets were dissolved in Milli-Q water and filtered through a 0.2 μm membrane before use. All stock solutions were stored at −20° C. Lipids were mixed in the molar ratios of interest in a glass vial. The CHCl₃ and/or methanol were evaporated using a gentle flow of nitrogen (N₂) while holding the vial almost horizontally until a lipid film of 2-10 mm was formed (2-10 mm). Subsequently, the vial was kept under vacuum for two hours to remove any remaining solvent. By adding Milli-Q, multilamellar vesicles were created at a concentration of 1 mg/ml. Unilamellar vesicles were formed by extruding the lipid vesicle mixture at least 15 times through a polycarbonate membrane (Whatman) with pore sizes of 0.1 μm. The extruding of the vesicle solution with this pore-size membrane resulted in vesicles of ~100 nm in diameter. The extruded vesicles were stored under argon in a fridge and used within two weeks after formation.

### Quartz-Crystal Microbalance Measurements

For flat QCM-D sensors, cleaning was performed using a 2 wt % sodium dodecyl sulfate (SDS) solution and thorough rinsing with Milli-Q. Activation was performed with 30 min UV/ozone treatment (for QCM-D sensors) using a Bioforce chamber (Nanosciences) or overnight incubation in 2% Hellmanex and again thorough Milli-Q rinsing.

### 96-Well Plate Experiments

A Janus liquid handler workstation (Perkin Elmer) was used to prepare SLBs in the 96-well plates in batches of 4 per run. The composition of the vesicle was kept the same for each experiment (1% Biotin, 0.2% TR). Prior to vesicle addition, the glass surface inside wells by addition of 200 μL of a 2 M NaOH (Merck) aqueous solution to each well. This activation was followed by throughout cleaning of the Wells with MQ water (15 times), before 100 μL/well of the vesicle solution (0.2 mg/mL in PBS) was added. 30 minutes of incubation at room temperature was followed by throughout cleaning of the wells with MQ water (15 times). During these cleaning steps 100 μL of fresh buffer was added to the well and after mixing, 100 UL was removed. This was followed by the 45 minutes incubation of a 0.2 μM SAv 488 and finally again the same cleaning procedure. After this process, the plate is removed from the liquid handler station and subsequent functionalization steps were performed by hand. Here, the same procedure of washing was performed with a micropipette.

### Fluorescence Microscopy

The individual wells of the 96 well plate were imaged using Olympus IX71 fluorescence microscope at ×20 magnification and 50% lamp intensity of a mercury lamp. The exposure times for TR-containing SLBs and Alexa Fluor 488-containing SAv were 300 ms and 1500 ms, respectively. The filter for TR had an excitation range of 510-550 nm and a lowpass filter allowed the emitted light as of 590 nm. Alexa Fluor 488 was excited in 460-490 nm range and the emissions were captured at 525 nm.

### Results

### Principle of Single DNA Detection by a 2D Confinement System

The current Example provides a 2D confinement system where a sensor surface is functionalized with a RNP complex and a fluorophore-quencher pair. Such a system utilizes the immobilization of both RNP and fluorophore-quencher to the sensor.

To benefit from the high trans-cleavage turnover of Cas effector proteins such as Cas12 or Cas13, immobilized RNPs preferably are able to reach thousands of reporter molecules, allowing the presence of a single target molecule be observed with a simple read-out method like fluorescence microscopy.

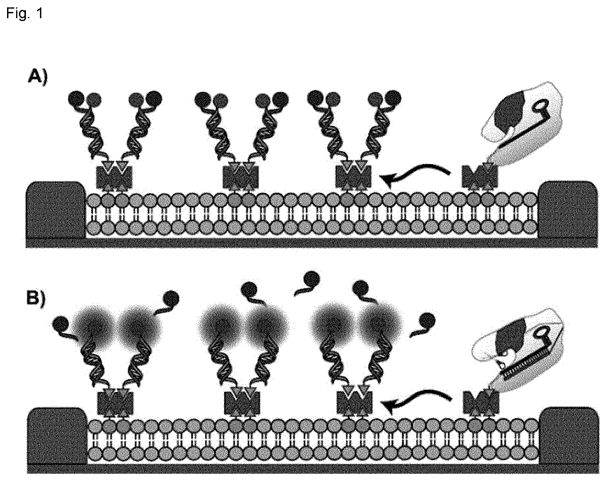

To take full advantage of the collateral cleavage activity of an immobilized RNP, it was decided to exploit supported lipid bilayers (SLBs) for both RNP and reporter immobilization. The unique properties of SLBs on hydrophilic substrates make it possible to create a 2D diffusion system where RNPs and reporters can (freely) diffuse. An SLB formed on a hydrophilic substrate is not static but allows lateral diffusion of the individual lipids across the substrate. A 2D-diffusion system of biomolecules can then be created by attaching biomolecules to these lipids. By immobilizing both CRISPR enzymes and fluorophore quencher pairs on lipids in the SLB, is system is created where CRISPR can access all fluorophore-quencher pairs via lateral diffusion, cleaving hundreds to thousands after successful target binding (FIG. 1).

It was found that the SLB membranes can be partitioned into wells (e.g. to form a so-called “corral structure”) by using metal lines on a glass/silica substrate, wherein each well contains preferably (on average) one RNP, or at least the same number of RNPs, so that the increase in fluorescence of each well can be related to the binding of a single target sequence. By Poisson statistics, the initial concentration of the target sequence is then calculated.

### Confirmation of SLB Formation and Reporter Immobilization

This Cas12a effector protein targets double-stranded DNA (dsDNA), and ssDNA will be trans-cleaved upon target binding-induced structural changes.

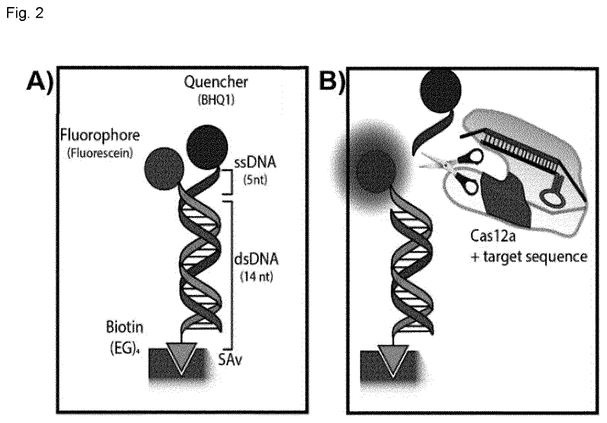

We confirmed the sensor assembly on a continuous hydrophilic interface. Because of its well-characterized and easy-to-use functionalization, streptavidin (SAv)-biotin chemistry was used to immobilize the reporter DNAs. As shown in FIG. 2, the sensor comprises a SLB with two types of lipids, one non-functionalized and one biotin-functionalized, which allows interactions with SAv. It was found that the molecular structure of SAv allows the immobilization of biotin-modified biomolecules on top of the SLB and which can freely diffuse across the lipid bilayer surface.

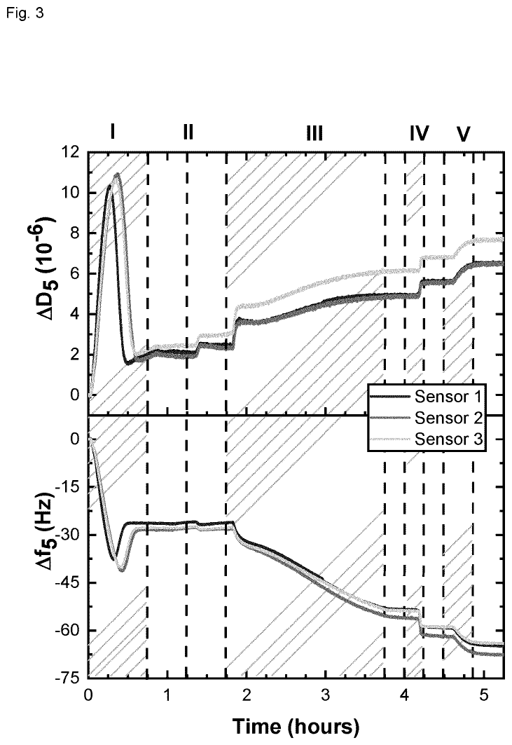

Quartz-crystal microbalance with dissipation (QCM-D) was used to monitor the formation of the SLB and subsequential functionalization steps. FIG. 3 shows an a typical QCM-D measurement, where each adsorption step on the surface is observed by a change in the frequency (Afs) and simultaneously in a change in the dissipation (ADs). After surface activation by UV-ozone, the silica-covered QCM sensor was mounted, after which SLB formation took place. For SLB formation, unilamellar vesicles with a diameter of 100 nm were prepared, consisting of 99% 1,2-dioleoyl-sn-glycero-3-phosphocholine (DOPC) and 1% of 1,2-dioleoyl-sn-glycero-3-phosphoethanolamineN-(biotinyl) (DOPE-biotin). On a hydrophilic, solid surface, these vesicles can adsorb, after which a spontaneous rupture of lipid vesicles results in SLB formation. The adsorption and subsequential rupture of the vesicles was confirmed by changes in both mass and rigidity (Δf₅=23±1 Hz and ΔD₅=2±0.5 Hz). Next, a solution of SAv (1 μM) flowed over the SLB. Adsorption of SAv onto the SLB was also confirmed by a clear, expected frequency shift (Δf₅=27±1 Hz).

The next step in the sensor assembly is immobilizing the “reporter.” As a trans-cleavage reporter, a fluorophore-quencher reporter was used similar to the one commonly used in solutions (Chen et al. Science (80-.), vol. 360, no. 6387, pp. 436-439, 2018). In a fluorophore-quencher pair, the fluorescence is suppressed by Föster resonance energy transfer between the fluorophore and quencher. This suppression is distance limited, meaning that cleavage of the nucleotide sequence between the fluorophore and quencher will restore the fluorescence signal. To immobilize the fluorophore-quencher pair on the surface, a short sequence (14 nt~4.5 nm) was designed that allows hybridization between a biotinylated ssDNA strand (containing the fluorophore) and a slightly longer ssDNA strand (with the quencher molecule). A 5 nt ssDNA strand separates the fluorophore and the quencher, allowing trans-cleavage by a RNP (also shown in FIG. 2).

Biotinylated ssDNA with a fluorescein fluorescent label (Δf₅=9±1 Hz) and complementary ssDNA with a quencher (Δf₅=7±2 Hz) were subsequently flowed over. Both the binding of the biotinylated DNA and the hybridization of the quencher DNA to the biotinylated DNA upon flushing with buffer were confirmed by QCM-D (FIG. 3, step IV and V, subsequently).

Besides QCM-D monitoring, also fluorescence microscopy was performed to monitor the sensor assembly. Here, the quenching of the fluorescence signal by the ssDNA containing the quencher was observed, which is an extra confirmation of successful sensor assembly. For fluorescent imaging, the protocol was slightly altered compared to the QCM-D measurements, enabling monitoring of SLB quality and streptavidin coverage optically. Black coated 96 well palates with flat glass bottom were cleaned and UVozone activated to enable SLB formation and fluorescence monitoring. Different from the functionalization protocol as performed during QCM measurements, an SLB was formed using 100 nm sized vesicles consisting of 98.8% DOPC, 1% DOPE-biotin and 0.2% (Texas Red) TR-DHPE and fluorescently labelled streptavidin was used.

### Immobilizing Cas12a on the SLB

Besides reporter sequence immobilization on the SLB, successful immobilization of Cas12a on the SLB is also needed. In the current example, Cas12a was not immobilized on the surface directly but via the RNP's crRNA sequence. The comparison of the two methods putatively shows that direct immobilization of Cas12a affects the and/or leads to less sensitivity or specificity in measurement of the target sequence. Without being bound by theory, it seems plasmid modification or unidirectional amine-related binding used in the direct method affects the conformation of the CRISPR protein.

An RNP comprises a CRISPR effector protein and a crRNA. This crRNA consists of a customizable component that defines the specificity and selectivity of target DNA and a non coding RNA part related to the association with the Cas12a protein. The non-coding part facilitates the association between crRNA and the effector protein by extensive hydrogen bond contacts and aromatic stacking between crRNA and the effector protein. In the current example, the crRNA is immobilized on the surface, followed by RNP complex formation on the surface. crRNA immobilization on the SLB uses a chemical modification of the RNA sequence. It is considered that for this particular Cas12a protein, 3′ and 5′ end extensions of the crRNA by either DNA or RNA could result in an amplified trans-cleavage activity. Furthermore, upon extension of the 3′ end of the crRNA with either DNA or RNA, an improved specificity of LbCas12a was found. Furthermore, several chemical modifications were added to both 3′ and 5′ end without affecting the trans-cleavage activity of the protein upon target binding.

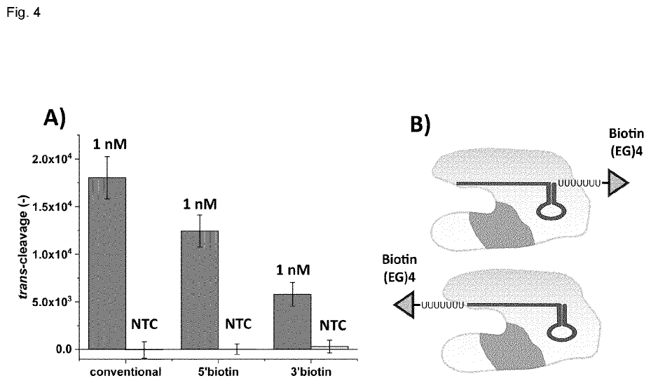

Although we exploit a different, commercially available Cas12a variant (AsCas12a ULTRA, Integrated DNA Technologies), we wanted to test whether we would observe similar trends in trans-cleavage activity upon modification of the crRNA on either the 3′ or 5′ end of the RNA sequence. Therefore, we added a spacer consisting of 7 uracil nucleotides (7 nt poly-U tail) between the crRNA and the chemical modification. Since we already use biotinylated lipids to immobilize the fluorophore-quencher pair, we decided to select biotin as a chemical modifier for crRNA for ease of use. The effect of this modified crRNA on the trans-cleavage activity of Cas12a compared to the conventional crRNA was tested in solution (FIG. 4). We observed significant trans-cleavage activity and continued with these crRNAs as an immobilization technique for Cas12a.

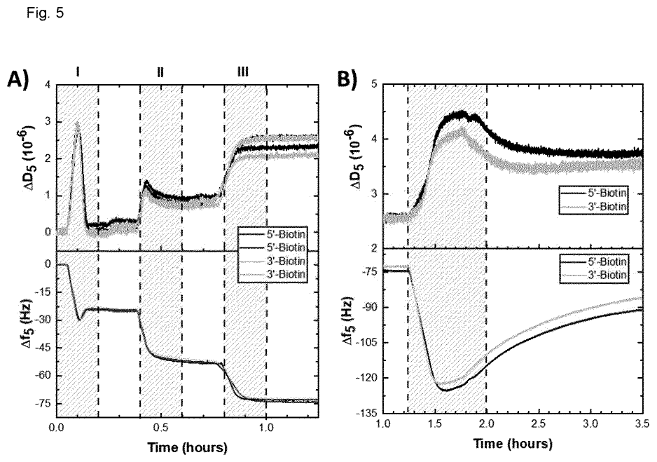

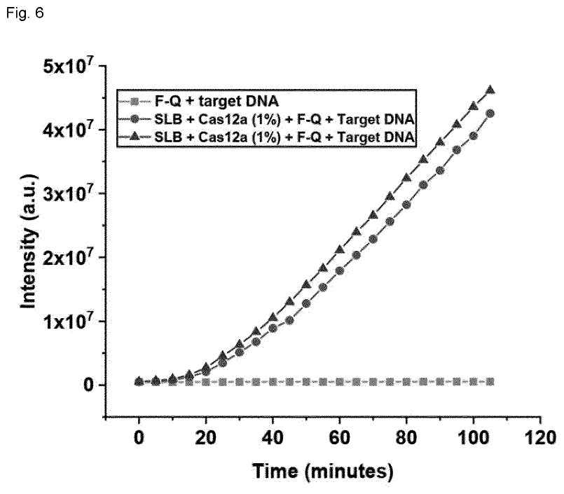

Assuming that every biotin-modified-crRNA binds a single Cas12a, a frequency change of about 20 Hz is expected during the Cas12a anchoring step. By flowing over a Cas12a solution, we observe a frequency change of ~14 Hz (Δf₅=13±1 Hz and Δf₅=15±2, FIG. 5B). With QCM-D measurements, the measured frequency change can be converted to an adsorbed mass by the Sauerbrey equation (Sauerbrey. Verwendung von Schwingquarzen zur Wägung dünner Schichten und zur Mikrowägung,” Zeitschrift für Phys., vol. 155, no. 2, pp. 206-222, 1959). The observed frequency shift corresponds to a surface density of ~2×10⁻¹² mole/cm². This lower frequency shift can be explained by steric hindrance of biotin-crRNAs bound to a single SAv, reducing the effective fraction of Cas12a that can bind crRNAs. By these experiments, the binding of the Cas12a protein in the presence of crRNA was confirmed. As the next step, the activity of the anchored Cas12a was tested by timed plate reader experiments, where target and fluorophore-quencher pairs were added in solution on top of the SLB, and the fluorescence intensity was measured over time (FIG. 6). This indeed showed trans-cleavage towards the fluorophore-quencher in solution in the presence of the anchored Cas12a.

The next step is to confirm the anchoring of Cas12a on the SLB. A similar frequency shifts for the binding of 3′ and 5′ biotinylated crRNA (Δf₅=19±0 Hz and Δf₅=19±1, FIG. 5A) to the SLB was observed. A frequency change of 19 Hz crRNA corresponds to a surface density of ~3×10⁻¹² mol/cm², which is in the same order of magnitude as the calculated biotin modified-lipid concentration expected on the surface (~2.3×10⁻¹² mol/cm²).

Similar results as above are achieved when the reporter and RNP are simultaneously immobilized on the same SLB, allowing detection of target DNA via the fluorescence signal.

### Confirming Detection of Cancer Biomarkers in Bladder Cancer

The marker panel GHSR/MAL as DNA methylation markers has high diagnostic performance for diagnosis bladder cancer using full void urine of patients (Hentschel et al. Clin Epigenetics. 2022 Feb. 5; 14 (1): 19). The current 2D confinement system is therefore used to detect the GHSR/MAL markers in patients with bladder cancer and healthy controls.

To this end, the reporter and RNP are simultaneously immobilized the on the same SLB. The GHSR/MAL target sequences and the homologous crRNA sequences (Alt-R® A.s. Cas12a crRNA) used were according to Table 1. A corral-type sensor is used, i.e. where each corral acts as an individual sensor for the different targets.

TABLE 1
Targeting sequences MAL/GHSR and crRNAs that target these sequences.

| Name | Target sequence (5′→3′) | crRNA sequence |
|---|---|---|
| GHSR | GGAAGTGCGAGATGGAACTG | /AltR1/rUrA rArUrU rUrCrU rArCrU rCrUrU |
| crRNA 1 | G (SEQ ID NO: 1) | rGrUrA rGrArU rGrGrA rArGrU rGrCrG |
| | | rArGrA rUrGrG rArArC rUrGrG/AltR2/ (SEQ |
| | | ID NO: 7) |
| GHSR | TCCCAACACATCCTCCGGCC | /AltR1/rUrA rArUrU rUrCrU rArCrU rCrUrU |
| crRNA 2 | G (SEQ ID NO: 2) | rGrUrA rGrArU rUrCrC rCrArA rCrArC |
| | | rArUrC rCrUrC rCrGrG rCrCrG/AltR2/ (SEQ |
| | | ID NO: 8) |
| GHSR | CCCCACCAACTCCCCCAAAG | /AltR1/rUrA rArUrU rUrCrU rArCrU rCrUrU |
| crRNA 3 | T (SEQ ID NO: 3) | rGrUrA rGrArU rCrCrC rCrArC rCrArA |
| | | rCrUrC rCrCrC rCrArA rArGrU/AltR2/ (SEQ |
| | | ID NO: 9) |
| MAL | CTGGCGGAACTGGACCACCG | /AltR1/rUrA rArUrU rUrCrU rArCrU rCrUrU |
| crRNA 1 | C (SEQ ID NO: 4) | rGrUrA rGrArU rCrUrG rGrCrG rGrArA |
| | | rCrUrG rGrArC rCrArC rCrGrC/AltR2/ (SEQ |
| | | ID NO: 10) |
| MAL | CGCTGGATGCGTTAATGTGC | /AltR1/rUrA rArUrU rUrCrU rArCrU rCrUrU |
| crRNA 2 | G (SEQ ID NO: 5) | rGrUrA rGrArU rCrGrC rUrGrG rArUrG |
| | | rCrGrU rUrArA rUrGrU rGrCrG/AltR2/ (SEQ |
| | | ID NO: 11) |
| MAL | AATTTGGGTCTCCTTCATTTT | /AltR1/rUrA rArUrU rUrCrU rArCrU rCrUrU |
| crRNA 3 | (SEQ ID NO: 6) | rGrUrA rGrArU rArArU rUrUrG rGrGrU |
| | | rCrUrC rCrUrU rCrArU rUrUrU/AltR2/ (SEQ |
| | | ID NO: 12) |

Table 2 shows crRNA sequences with biotin modifications used for immobilization (as ordered from IDT (Coralville, Iowa, USA).

TABLE 2
crRNA sequences with biotin modifications

| Name | crRNA sequence |
|---|---|
| MALcrRNA_ex7_b3′ (5′→3′) | rUrArA rUrUrU rCrUrA rCrUrC rUrUrG rUrArG rArUrC |
| | rGrCrU rGrGrA rUrGrC rGrUrU rArArU rGrUrG rCrGrA |
| | rUrUrA rUrUrA/3BioTEG/ (SEQ ID NO: 13) |
| MALcrRNA_ex7_b3′ (5′→3′) | /5Biotin TEG/rUrU rArUrU rArUrU rArArU rUrUrC |
| | rUrArC rUrCrU rUrGrU rArGrA rUrCrG rCrUrG rGrArU |
| | GrCrG rUrUrA rArUrG rUrGrC rG (SEQ ID NO: 14) |

It is confirmed that cancer patients and healthy markers can be discriminated by this method with appropriate sensitivity and specificity. The detection limit could be lowered to the low pM range or even fM range. Similar results are obtained with other Cas effector protein with a collateral trans-cleavage activity, such as Cas13. It is found that by changing the ratio of crRNA to ssDNA, the ratio between the RNP and reporter can be changed and optimized, e.g. to a ratio between 1:100-1:1E8.

What is claimed is:

1. A device for detecting at least one target nucleic acid sequence, wherein the device comprises a surface which carries
at least one activatable protein which is activated in the presence of the at least one target nucleic acid sequence;
one or more reporter constructs which provide a detectable signal upon activation of the activatable protein,
wherein the at least one activatable protein and/or the one or more reporter constructs are immobilized, optionally via a linker, to one or more moieties capable of moving along the surface.

2. The device according to claim 1, wherein the one or more moieties capable of moving along the surface are amphipathic molecules, preferably cholesterol, or hydrophobic molecules, preferably lipids.

3. The device according to claim 1, wherein the surface carries a lipid monolayer or lipid bilayer, preferably a lipid bilayer comprising a first layer of lipids and a second layer of lipids and wherein the one or more moieties capable of moving along the surface are comprised in the first and/or second layer of lipids.

4. The device according to claim 1, wherein activation of the at least one activatable protein provides said protein of trans-cleavage activity and wherein the trans-cleavage activity cleaves at least one single stranded nucleic acid sequence in the one or more reporter constructs thereby providing a detectable signal.

5. The device according to claim 1, wherein the at least one activatable protein is supplemented with an RNA sequence complementary to the at least one target nucleic acid sequence, and preferably the RNA sequence complementary to the nucleic acid target sequence is a CRISP RNA (crRNA).

6. The device according to claim 1, wherein the at least one activatable protein is a Cas protein, preferably a ribonucleotide protein (RNP), more preferably Cas12, Cas13, or Cas14.

7. The device according to claim 1, wherein the detectable signal is one or more of fluorescence, luminescence, electrical activity, electrochemical activity, chemical activity, charge, enzymatic activity, radioactivity, colorimetry, mass, mass change, optical shift, and magnetism.

8. The device according to claim 1, wherein the one or more reporter constructs comprise a fluorophore and a quencher of the fluorophore separated by at least one single stranded nucleic acid sequence cleavable by trans-cleavage activity.

9. The device according to claim 1, wherein the at least one activatable protein and/or the one or more reporter constructs are immobilized to one or more moieties capable of moving along the surface via a linker, wherein the linker is biotin and/or a biotin-binding moiety.

10. The device according to claim 1, wherein the at least one activatable protein is immobilized to one or more moieties capable of moving along the surface via a linker, wherein the linker is an RNA sequence complementary to the at least one target nucleic acid sequence.

11. The device according to claim 1, wherein the number of activatable proteins and the number of reporter constructs is in a ratio of between 1:200-1:50000.

12. The device according to claim 1, wherein the surface is hydrophilic or hydrophobic.

13. The device according to claim 1, wherein the surface comprises at least two separate partitions each comprising
the at least one activatable protein which is activated in the presence of the at least one target nucleic acid sequence;
the one or more reporter constructs which provide a detectable signal upon activation of the at least one activatable protein,
wherein the at least one activatable protein and/or the one or more reporter constructs are immobilized in each partition, optionally via a linker, to one or more moieties capable of moving along the surface.

14. The device according to claim 13, wherein the at least two separate partitions are formed by a physical and/or hydrophobic enclosure thereby preventing exchange of the at least one activatable protein and/or the one or more reporter constructs between the at least two partitions.

15. A method for detecting at least one target nucleic acid sequence, the method comprising
i. applying a liquid sample comprising the at least one target nucleic acid sequence to the surface of the device according to any one of the previous claims; and
ii. detecting activation of the activatable protein, preferably by measuring the detectable signal provided by one or more reporter constructs.

16. The method according to claim 15, wherein the liquid sample is a body fluid of a subject preferably one or more of urine, blood, saliva, serum, blood plasma, interstitial fluid, synovial fluid, transudate, pus, breast milk, and semen, more preferably urine.

17. The method according to claim 15, wherein the liquid sample is a body fluid of subject which is a cancer patient and/or wherein the at least one target nucleic acid sequence comprises a cancer biomarker.

18. The method according to claim 15, wherein the at least one target nucleic acid sequence is present in the liquid sample in an amount of 1×10⁻¹⁸-1×10⁻¹⁰ M.

19. (canceled)


SEQUENCE LISTING (14 sequences)

SEQ ID NO: 1 - DNA, 21 bp, unidentified
  source 1..21: mol_type = unassigned DNA
ggaagtgcgagatggaactgg

SEQ ID NO: 2 - DNA, 21 bp, unidentified
  source 1..21: mol_type = unassigned DNA
tcccaacacatcctccggccg

SEQ ID NO: 3 - DNA, 21 bp, unidentified
  source 1..21: mol_type = unassigned DNA
ccccaccaactcccccaaagt

SEQ ID NO: 4 - DNA, 21 bp, unidentified
  source 1..21: mol_type = unassigned DNA
ctggcggaactggaccaccgc

SEQ ID NO: 5 - DNA, 21 bp, unidentified
  source 1..21: mol_type = unassigned DNA
cgctggatgcgttaatgtgcg

SEQ ID NO: 6 - DNA, 21 bp, unidentified
  source 1..21: mol_type = unassigned DNA
aatttgggtctccttcatttt

SEQ ID NO: 7 - RNA, 82 nt, unidentified
  source 1..82: mol_type = unassigned RNA
rtrarartrtrtrcrtrarcrtrcrtrtrgrtrargrartrgrgrarargrtrgrcrgrargrartrgrgrararcrtrgrg

SEQ ID NO: 8 - RNA, 82 nt, unidentified
  source 1..82: mol_type = unassigned RNA
rtrarartrtrtrcrtrarcrtrcrtrtrgrtrargrartrtrcrcrcrararcrarcrartrcrcrtrcrcrgrgrcrcrg

SEQ ID NO: 9 - RNA, 82 nt, unidentified
  source 1..82: mol_type = unassigned RNA
rtrarartrtrtrcrtrarcrtrcrtrtrgrtrargrartrcrcrcrcrarcrcrararcrtrcrcrcrcrcrararargrt

SEQ ID NO: 10 - RNA, 82 nt, unidentified
  source 1..82: mol_type = unassigned RNA
rtrarartrtrtrcrtrarcrtrcrtrtrgrtrargrartrcrtrgrgrcrgrgrararcrtrgrgrarcrcrarcrcrgrc

SEQ ID NO: 11 - RNA, 82 nt, unidentified
  source 1..82: mol_type = unassigned RNA
rtrarartrtrtrcrtrarcrtrcrtrtrgrtrargrartrcrgrcrtrgrgrartrgrcrgrtrtrarartrgrtrgrcrg

SEQ ID NO: 12 - RNA, 82 nt, unidentified
  source 1..82: mol_type = unassigned RNA
rtrarartrtrtrcrtrarcrtrcrtrtrgrtrargrartrarartrtrtrgrgrgrtrcrtrcrcrtrtrcrartrtrtrt

SEQ ID NO: 13 - RNA, 96 nt, unidentified
  source 1..96: mol_type = unassigned RNA
rtrarartrtrtrcrtrarcrtrcrtrtrgrtrargrartrcrgrcrtrgrgrartrgrcrgrtrtrarartrgrtrgrcrgrartrtrartrtra

SEQ ID NO: 14 - RNA, 96 nt, unidentified
  source 1..96: mol_type = unassigned RNA
rtrtrartrtrartrtrarartrtrtrcrtrarcrtrcrtrtrgrtrargrartrcrgrcrtrgrgrartrgrcrgrtrtrarartrgrtrgrcrg